(19) United States
(12) Patent Application Publication
(10) Pub. No.: US 2006/0276864 A1
(43) Pub. Date: Dec. 7, 2006

(54) Systems and methods for sensing patient temperature in temperature management system

(75) Inventor: Kenneth A. Collins, Mission Viejo, CA (US)

Correspondence Address: ROGITZ & ASSOCIATES, 750 B STREET, SUITE 3120, SAN DIEGO, CA, 92101, US

(73) Assignee: ALSIUS CORPORATION

(21) Appl. No.: 11/145,103

(22) Filed: Jun. 3, 2005

(51) Int. Cl. A61F 7/00; A61F 7/12

(52) U.S. Cl. 607/105; 607/113

(57) ABSTRACT

Various methods are disclosed for measuring patient temperature for input as a feedback signal into a temperature management system.

### I. FIELD OF THE INVENTION

The present invention relates generally to patient temperature management.

### II. BACKGROUND OF THE INVENTION

Intravascular catheters have been introduced for controlling patient temperature. Typically, a coolant such as saline is circulated through an intravascular heat exchange catheter, which is positioned in the patient's bloodstream, to cool or heat the blood as appropriate for the patient's condition. The coolant is warmed or cooled by a computer-controlled heat exchanger that is external to the patient and that is in fluid communication with the catheter.

For example, intravascular heat exchange catheters can be used to combat potentially harmful fever in patients suffering from neurological and cardiac conditions such as stroke, subarachnoid hemorrhage, intracerebral hemorrhage, cardiac arrest, and acute myocardial infarction, or to induce therapeutic hypothermia in such patients. Further, such catheters can be used to rewarm patients after, e.g., cardiac surgery or for other reasons. Intravascular catheters afford advantages over external methods of cooling and warming, including more precise temperature control and more convenience on the part of medical personnel.

The following U.S. patents, all of which are incorporated herein by reference, disclose various intravascular catheters/systems/methods: 6,419,643, 6,416,533, 6,409,747, 6,405,080, 6,393,320, 6,368,304, 6,338,727, 6,299,599, 6,290,717, 6,287,326, 6,165,207, 6,149,670, 6,146,411, 6,126,684, 6,306,161, 6,264,679, 6,231,594, 6,149,676, 6,149,673, 6,110,168, 5,989,238, 5,879,329, 5,837,003, 6,383,210, 6,379,378, 6,364,899, 6,325,818, 6,312,452, 6,261,312, 6,254,626, 6,251,130, 6,251,129, 6,245,095, 6,238,428, 6,235,048, 6,231,595, 6,224,624, 6,149,677, 6,096,068, 6,042,559.

Surface cooling may be less optimally used. For example, externally applied cooling pads are disclosed in U.S. Pat. Nos. 6,827,728, 6,818,012, 6,802,855, 6,799,063, 6,764,391, 6,692,518, 6,669,715, 6,660,027, 6,648,905, 6,645,232, 6,620,187, 6,461,379, 6,375,674, 6,197,045, and 6,188,930 (collectively, “the external pad patents”), all of which are incorporated herein by reference.

Regardless of the modality of cooling, patient temperature must be measured to provide a feedback signal to the controller.

### SUMMARY OF THE INVENTION

A system for controlling patient temperature includes a heat transfer member engageable with a human patient and a temperature control system engageable with the heat transfer member to circulate working fluid therethrough. A patient temperature sensor generates a signal that represents patient temperature and sends the signal to the control system. The sensor is a temperature sensor positioned against the skin of a patient above the temporal artery and covered with a thermally insulative biocompatible shield such as, e.g., a polymeric gel, or it can be a temperature sensor located at a sublingual region of a support such as, e.g., a mouth guard located in the patient's mouth. The heat transfer member may be a closed loop catheter or a heat transfer pad. In non-limiting embodiments the temperature sensor is associated with a wireless transmitter which wirelessly transmits the signal to the control system. In other embodiments the sensor is mounted on a mouth guard and is connected to an electrical wire ending in a connector that is configured for mechanically engaging the control system.

In another aspect, a patient temperature sensor system includes a mouth guard defining a sublingual region, and a temperature sensor disposed on the mouth guard at the sublingual region. Means are provided for sending a signal from the temperature sensor to a control system.

In still another aspect, a method for generating a signal representative of patient temperature includes positioning a temperature sensor against a patient over a temporal artery and then covering the sensor with a thermally insulative biocompatible shield. A signal from the sensor is sent to a control system.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a schematic diagram showing two modalities of temperature maintenance in a patient, along with an apparatus for quickly reducing patient temperature;

FIG. 2 is a perspective view of a first patient temperature sensor, using a sensor placed on the patient over the temporal artery, showing for illustration blood vessels that normally would be hidden under the skin;

FIG. 3 is a top view of a second patient temperature sensor incorporated on the sublingual side of a mouth guard; and

FIG. 4 is a front view of an alternate embodiment of the second patient temperature sensor incorporated on the sublingual side of a mouth guard.

### DETAILED DESCRIPTION OF THE PREFERRED EMBODIMENT

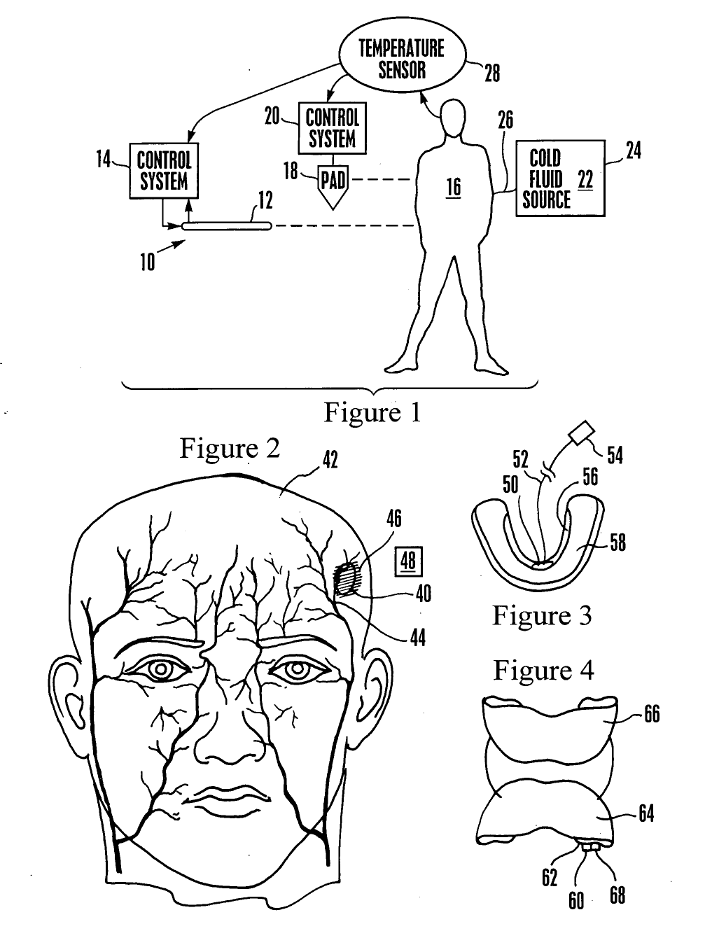

Referring initially to FIG. 1, a system is shown, generally designated 10, that includes a heat exchange catheter 12 that is in fluid communication with a catheter temperature control system 14.

In accordance with present principles, the system 10 can be used to induce therapeutic hypothermia in a patient 16 using a catheter in which coolant circulates in a closed loop, such that no coolant enters the body. While certain preferred catheters are disclosed below, it is to be understood that other catheters can be used in accordance with present principles, including, without limitation, any of the catheters disclosed in the following U.S. patents, all incorporated herein by reference: U.S. Pat. Nos. 5,486,208, 5,837,003, 6,110,168, 6,149,673, 6,149,676, 6,231,594, 6,264,679, 6,306,161, 6,235,048, 6,238,428, 6,245,095, 6,251,129, 6,251,130, 6,254,626, 6,261,312, 6,312,452, 6,325,818, 6,409,747, 6,368,304, 6,338,727, 6,299,599, 6,287,326, 6,126,684. The catheter 12 may be placed in the venous system, e.g., in the superior or inferior vena cava.

Instead of or in addition to the catheter 12, the system 10 may include one or more pads 18 that are positioned against the external skin of the patient 16 (only one pad 18 shown for clarity). The pad 18 may be, without limitation, any one of the pads disclosed in the external pad patents. The temperature of the pad 18 can be controlled by a pad controller 20 in accordance with principles set forth in the external pad patents to exchange heat with the patient 16, including to induce therapeutic mild or moderate hypothermia in the patient in response to the patient presenting with, e.g., cardiac arrest, myocardial infarction, stroke, high intracranial pressure, traumatic brain injury, or other malady the effects of which can be ameliorated by hypothermia.

To cool the patient while awaiting engagement of the catheter 12 and/or pad 18 with the patient, cold fluid 22 in a cold fluid source 24 may be injected into the patient and in particular into the patient's venous system through a pathway 26. Without limitation, the pathway 26 may an IV line, the source 24 may be an IV bag, and the fluid 22 may be chilled saline, e.g., saline at the freezing point or slightly warmer. Or, the source may be a syringe, and the saline can be injected directly into the bloodstream of the patient.

A temperature sensor 28 senses patient temperature and sends a feedback signal to one or both of the control systems 14, 20.

Now referring to FIG. 2, a temperature sensor of the present invention can be seen. A temperature sensor 40, which may be small and flat, is positioned against the skin of a patient 42 above the temporal artery 44. The sensor 40 may be any suitable temperature sensing device, including but not limited to a thermocouple, an infrared sensor, a resistance temperature device (RTD), a chemical expansion device, etc. Regardless of the type of sensor, it is covered by a thermally insulative biocompatible shield 46, which may be a polymeric gel. The sensor 40 thus may be placed against the skin over the temporal artery and then covered with the shield 46 to prevent the environment from heating or cooling the sensor 40 apart from heating or cooling of the sensor induced by the patient, so that the signal from the sensor accurately represents patient temperature without the need to use an ambient temperature measurement as a compensation signal. A wire (not shown) can connect the sensor 40 to one of the above control systems, or the sensor can send its signal to a wireless transmitter 48 that may be incorporated in the same housing as the sensor 40 for transmitting the signal to a wireless receiver in the relevant control system.

FIG. 3 shows a temperature sensor 50 that is connected to an electrical wire 52 which ends in a connector 54, and the connector 54 can be connected to one of the control systems above for sending the signal generated by the sensor to the control system. The sensor 50 may be any suitable temperature sensor that generates an electrical signal which is useful by a processor in the control system, meaning that the sensor is not a substance that represents temperature by changing color and further that the sensor is not distributed throughout the mouth guard, but rather is embedded in a single location of the mouth guard. As shown in FIG. 3, it is mounted on sublingual side 56 of a mouth guard 58 that is configured for covering the lower teeth of a patient. Thus, the sensor 50 generates a signal representative of the sublingual temperature of the patient. The sensor 50 may be embedded in the mouth guard material near the surface thereof or adhered to the mouth guard or otherwise mounted on the mouth guard.

FIG. 4 shows a temperature sensor 60 mounted on the sublingual side 62 of a mouth guard 64, in all essential respects identically to the invention shown in FIG. 3 except that the mouth guard 64 may be hinged to an upper mouth guard 66 and instead of sending the temperature signal to a control system via a wire, a wireless transmitter 68 is positioned on the mouth guard 64 and is electrically connected to the sensor 60 for wirelessly transmitting the temperature signal to one of the control systems disclosed above.

While the particular SYSTEMS AND METHODS FOR SENSING PATIENT TEMPERATURE IN TEMPERATURE MANAGEMENT SYSTEM as herein shown and described in detail is fully capable of attaining the above-described objects of the invention, it is to be understood that it is the presently preferred embodiment of the present invention and is thus representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more”. All structural and functional equivalents to the elements of the above-described preferred embodiment that are known or later come to be known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. §112, sixth paragraph, unless the element is expressly recited using the phrase “means for” or, in the case of a method claim, the element is recited as a “step” instead of an “act”.

What is claimed is:

1. A system for controlling patient temperature, comprising:
a heat transfer member engageable with a human patient;
a temperature control system engageable with the heat transfer member to circulate working fluid therethrough; and
at least one patient temperature sensor generating a signal representative of patient temperature and sending the signal to the control system, the sensor being selected from the group consisting of: a temperature sensor positioned against the skin of a patient over the temporal artery and covered with a thermally insulative biocompatible shield, and a temperature sensor located at a sublingual region of a support disposed in the patient's mouth.

2. The system of claim 1, wherein the heat transfer member is a closed loop catheter.

3. The system of claim 1, wherein the heat transfer member is at least one heat transfer pad.

4. The system of claim 1, wherein the temperature sensor is associated with a wireless transmitter wirelessly transmitting the signal to the control system.

5. The system of claim 1, wherein the temperature sensor is positioned against the skin of a patient above the temporal artery and covered with a polymeric gel.

6. The system of claim 1, wherein the sensor is mounted on a mouth guard and is connected to an electrical wire ending in a connector configured for mechanically engaging the control system.

7. A patient temperature sensor system, comprising:
a mouth guard defining a sublingual region;
a temperature sensor disposed on the mouth guard at the sublingual region; and
means for sending a signal from the temperature sensor to a control system.

8. The system of claim 7, wherein the means for sending is a wireless transmitter.

9. The system of claim 7, wherein the means for sending is a wire.

10. A method for generating a signal representative of patient temperature, comprising:
positioning a temperature sensor against a patient over a temporal artery;
covering the sensor with a thermally insulative biocompatible shield; and
sending a signal from the sensor to a control system.

11. The method of claim 10, wherein the sending act is undertaken wirelessly.

12. The method of claim 10, wherein the shield is a polymer.

13. The method of claim 12, wherein the polymer is in gel form.
